(19) United States
(12) Patent Application Publication
(10) Pub. No.: US 2022/0041651 A1
(43) Pub. Date: Feb. 10, 2022

(54) BIOINSPIRED HIGHLY THERMO-SUSTAINABLE PACKINGS WITH USES THEREOF

(71) Applicant: Ramot at Tel-Aviv University Ltd., Tel Aviv (IL)

(72) Inventors: Kai TAO, Tel Aviv (IL); Sigal LAZAR, Tel Aviv (IL); Sharon GILEAD, Tel Aviv (IL); Ehud GAZIT, Tel Aviv (IL)

(21) Appl. No.: 17/393,529

(22) Filed: Aug. 4, 2021

Related U.S. Application Data
(60) Provisional application No. 63/061,254, filed on Aug. 5, 2020.

(51) Int. Cl. C07K 5/065

(52) U.S. Cl. CPC C07K 5/06078

(57) ABSTRACT

A thermally stable composition having at least one aromatic cyclic di-peptide is provided having a thermal sustainability of up to 680 Kelvin. The thermally stable compositions can be used in high temperature applications.

### CROSS REFERENCE TO RELATED APPLICATIONS

This application claims the benefit of Provisional U.S. Patent Application Ser. No. 62/529,506, entitled STABLE DIPEPTIDE ASSEMBLIES, filed Aug. 5, 2020, which is hereby incorporated in its entirety by reference.

### FIELD OF THE INVENTION

The present invention relates to bioinspired supramolecular semiconductors, and more particularly, peptide semiconductive assemblies.

### BACKGROUND OF THE INVENTION

Bioinspired supramolecular semiconductors, specifically peptide semiconductive assemblies, have shown promising potential for optical and electronic applications, especially in biological environments and at bio-machine interfaces, due to their intrinsic biocompatibility and ease of engineerability. However, peptide molecules have intrinsic thermal fragility. The design of a self-assembling semiconductive system with high thermal sustainability comprised of simple peptide molecules has so far not been demonstrated, and correspondingly, the underlying physical mechanisms have not yet been investigated

Accumulating studies demonstrate that aromatic linear-dipeptides, with the representative model of diphenylalanine (FF), can self-assemble into nanostructures with remarkable physiochemical features, such as optic, electrical, piezoelectric (including ferroelectric and pyroelectric) properties. It has been shown that the supramolecular morphologies and properties can be easily modified by amino acids substitutions, covalent conjugation or co-assembly with external moieties. For example, upon substitution of one F with tryptophan (W), self-assembling FW nanostructures present a smaller bandgap of 2.25 eV, compared to 3.25 eV of FF nanotubes, thus showing improved conductive and photolumine scent properties. See, for example, Tao et al. Science. 2017 Nov. 17; 358(6365): doi: 10.1 126/science. aam9756.

Recent studies revealed that cyclo-dipeptides with backbones of 2,5-diketopiperazine configurations, derived from dehydration condensation of linear dipeptides, self-assemble into photoluminescent nanostructures different from their linear counterparts [Lee, J. S. et al. Angew. Chem. Int. Ed. 50, 1164-1167 (2011); Yan et al. Angew. Chem. Int. Ed. 50, 11186-11191 (2011); Manchineella, S. & Govindaraju, T. ChemPlusChem 82, 88-106 (2016); and Amdursky, N. et al. Biomacromolecules 12, 1349-1354 (2011)].

Cyclic-peptides derived from amino acid residues carrying complexing side chain substituents, such as imidazole, carboxylate or thioether groups, can be used as models to mimic the coordination of metal ions in enzymes. [Ma et al. J. Am. Chem. Soc. 2014, 136 (51), 17734-17737; Clark et al. J. Am. Chem. Soc. 1998, 120 (4), 651-656; Bellezza et al. Trends in Molecular Medicine 2014, 20 (10), 551-558; Anderson et al. Coordination Chemistry Reviews 2017, 349, 102-128; Zou et al. Chemical Society Reviews 2015, 44 (15), 5200-5219; and Mannini et al. ACS Chemical Neuroscience 2018, 9 (12), 2959-2971].

Cyclic-dipeptides are highly tunable due to hydrogen bonding capabilities of the skeleton and other noncovalent interactions, that can be used to engineer artificial multifunctional scaffolds [Montenegro et al. Accounts of Chemical Research 2013, 46 (12), 2955-2965; Mantion et al. J. Am. Chem. Soc. 2008, 130 (8), 2517-2526].

Additional background art includes WO 2010/038228; Gazit, E. Peptide nanostructures: aromatic dipeptides light up. Nature Nanotechnol. 11, 309-310 (2016); Tao, K. et al. Nature Commun. 9, 3217 (2018); Tao, K. Peptide Semiconductor Times Are Coming. Go(dot)nature(dot)com/2MgoxSF; Kai Tao, Ehud Gazit. Aromatic peptide assemblies as bio inspired supramolecular semiconductors. Peptide Self-Assembly: Biology, Chemistry, Materials and Engineering, Beijing, China, August 2018 (Poster); Kai Tao, Ehud Gazit. Aromatic cyclo dipeptide self-assemblies with quantum confined photoluminescence from visible to near-infrared ranges. The 5th BioE12018 International Winterschool on Bioelectronics, Kirchberg in Tirol, Austria, March 2018 (Poster); Tao et al. Science 358, eaam9756 (2017); Tao et al., Mater Today (Kidlington) Author manuscript; available in PMC 2019 Nov. 12; Yuan et al., Research (Wash DC) 2019; 2019:9025939 doi: 10.34133/2019/9025939; and Tao et al., Adv. Funct. Mater. 2020, 1909614, all of which are incorporated by reference as of fully set forth herein.

### SUMMARY OF THE INVENTION

It is an object of the present invention to provide biologically thermo-sustainable materials for applications in heat-sensitive fields, such as body temperature detection and heat harvesting in bio-integrated microdevices, and waste heat utilization for energy generation.

It is further an object of the present invention to provide thermo-sustainable peptide assemblies that can be used as bio-inspired, eco-friendly alternatives for state-of-the-art inorganic or artificially made organic counterparts in conventional heat-sensitive fields.

According to an aspect of some embodiments of the present invention, there is provided a thermally stable composition comprising at least one aromatic cyclic di-peptide, wherein the composition has a thermal sustainability of up to 680 Kelvin.

In some embodiments, at least one aromatic cyclic dipeptide is a simple aromatic cyclic dipeptide. In certain of those embodiments, the at least one simple aromatic dipeptide is a cyclo-ditryptophan.

In certain embodiments, the composition has a thermal sustainability of about 580 Kelvin to about 680 Kelvin. In additional embodiments, the composition has a thermal sustainability of about 630 Kelvin to about 680 Kelvin. In further embodiments, the composition has a thermal sustainability of about 650 Kelvin to about 680 Kelvin.

In some embodiments, the at least one aromatic cyclic dipeptide comprises a plurality of aromatic cyclic dipeptide molecules forming a self-assembled structure.

In certain embodiments, the composition has a thermal quenching activity energy of up to 0.11 eV. In additional embodiments, the composition has a thermal quenching activity energy of about 0.03 eV to about 0.11 eV.

In some embodiments, the at least one aromatic cyclic dipeptide comprises at least one indole ring. In certain of those embodiments, the at least one indole ring comprises one or more substituent groups that modulate said thermal sustainability of the composition.

A semiconductor system is also provided, comprising a self-assembled structure formed of one or more cyclic peptides, wherein said at least one of said one or more cyclic peptides is an aromatic cyclic dipeptide, wherein said self-assembled structure is thermally stable at up to 680 Kelvin.

In some embodiments, the one or more cyclic peptides is a cyclo-ditryptophan.

In certain embodiments, the self-assembled structure has a thermal sustainability of about 580 Kelvin to about 680 Kelvin.

In some embodiments, the self-assembled structure has an average size of less than 100 nm at least in one dimension or cross-section.

An in-vivo implantable system is further provided, comprising a self-assembled structure formed of one or more aromatic cyclic dipeptides, wherein the self-assembled structure is thermally stable at up to 680 Kelvin, and wherein the implantable system is configured to self-charge.

In some embodiments, the one or more aromatic cyclic dipeptides is a cyclo-ditryptophan.

A thermally stable optical system is also provided, comprising a self-assembled structure formed of one or more aromatic cyclic dipeptides, wherein the self-assembled structure is thermally stable at up to 680 Kelvin.

In certain embodiments, the one or more aromatic cyclic dipeptides is a cyclo-ditryptophan.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B present a thermal sustainability characterization of an exemplary cyclo-dipeptide according to some embodiments of the present invention, cyclo-WW. FIG. 1A shows TGA curves of cyclo-WW and linear-WW. FIG. 1B shows comparison of the thermal sustainability of state-of-the-art semiconductive constituents.

FIG. 2 illustrates crystallographic characterization of the cyclo-WW crystals. Section (a) shows a SEM image of needle-like crystals of the cyclo-WW. Section (b) shows crystal structure of the cyclo-WW crystals along the crystal axis direction. For clarity, the side-chain indole rings are blurred. Section (c) shows the crystal structure of the cyclo-WW crystals along the crystal transection.

FIG. 3A illustrates a temperature-dependent emission spectrum of the of the cyclo-WW crystals excited at 370 nm. FIG. 3B shows a normalized emission spectrum shown in FIG. 3A. FIG. 3C shows a temperature-dependent normalized integrated intensity of the zero-phonon emission line at 445 nm (77 Kelvin). FIG. 3D shows FWHM of zero-phonon emission line evolution versus temperature.

FIGS. 4A-4F illustrate a microscopic mechanism of the high thermo-sustainability of the cyclo-WW crystals. FIG. 4A shows the interaction fractions inside the cyclo-WW crystals versus the temperature. FIGS. 4B-4D illustrate snapshots of part of the system at 300 Kelvin, 500 Kelvin, and 585 Kelvin. FIG. 4E shows the volume of the simulation box plotted versus the temperature. FIG. 4F is a schematic illustration showing the dynamic process of the destruction of cyclo-WW crystal structures upon temperature increase.

FIG. 5 illustrates a statistical distribution of temperature-dependent conductive resistance of the cyclo-WW crystals. The error bars represent the standard derivations.

FIGS. 6A-6B illustrate mechanical characterization of an exemplary cyclo-dipeptide according to some embodiments of the present invention, cyclo-WW. FIG. 6A shows Young's modulus and FIG. 6B shows Point stiffness of cyclo-WW crystals.

FIG. 7 illustrates crystallographic characterization of c-Ww crystals. Section (a) shows a SEM image of needle-like crystals assembled by c-Ww. Section (b) shows a crystal structure along the axis direction of the. For clarity, the side-chain indole rings are blurred. Section (c) shows a crystal structure along the transection of the crystal. The hydrogen bonding and aromatic interactions are labelled near their respective locations.

FIG. 8 shows TGA curves of c-Ww (shown in red) and l-Ww (shown in blue). The TGA curve of cyclo-WW (shown in black) was extracted from FIG. 3 for comparison.

FIGS. 9A-9B illustrate temperature-dependent conductivity of an exemplary cyclo-dipeptide according to some embodiments of the present invention, cyclo-WW. FIG. 9A shows voltage-current curves of cyclo-WW crystals at different temperatures. FIG. 9B shows resistance distribution calculated from FIG. 9A.

### DETAILED DESCRIPTION OF THE INVENTION

In the following description, numerous details are set forth for the purpose of explanation. However, one of ordinary skill in the art will realize that the invention may be practiced without the use of these specific details.

The invention comprises bio-inspired self-assembled crystals formed by a cyclic aromatic dipeptide, in one of the preferred embodiments, a cyclo-tryptophan-tryptophan (cyclo-WW), that crystallize into supramolecular semiconductors with thermal-sustainably up to 680 Kelvin. The present inventors have discovered that the non-covalent interactions underlie the driving forces of the thermal stability, generating a small exciton binding energy of only 0.29 eV, resulting in a long-wavelength emission in the visible light region, and high thermal quenching activity energy of up to 0.11 eV, five-fold higher than that of the well-studied diphenylalanine peptide (0.021 eV). The contributing forces comprise predominantly of aromatic interactions, followed by hydrogen bonding between peptide molecules and, to a lesser extent, water-mediated associations. The thermal sustainability of the bioinspired semiconductive architectures results in a 93% reduction of resistance from 320 K to 440 Kelvin.

A self-assembled structure or crystals as described herein is composed of a plurality of molecules, that is, peptide molecules as described herein, which assemble together to form a three-dimensional (e.g., at least partially ordered) structure. The peptide molecules are linked to one another by non-covalent bonds, preferably via π-π aromatic interactions.

The self-assembled structure is typically formed spontaneously (self-assemble) when the plurality of molecules (e.g., cyclic peptides) are contacted together and subjected to conditions that allow self-assemble to occur. Such conditions typically include contacting the molecules in the presence of a suitable solvent, at a concentration that allows self-assemble to occur, as described in further detail hereinafter.

In some of any of the embodiments described herein, the self-assembled structure is a self-assembled nanostructure, that is, a structure that has an average size of less than 1 micrometer, or less than 500 nm, or less than 100 nm, of at least one dimension or cross-section thereof.

According to some embodiments, a cyclic peptide is independently a cyclic short peptide which comprises up to 10 amino acid residues, preferably from 2 to 6 amino acid residues.

In some of any of the respective embodiments, at least a portion, or each, of the plurality of cyclic peptides comprises cyclic peptides of 2 to 10 amino acid residues, optionally from 2 to 9 amino acid residues, optionally from 2 to 8 amino acid residues, optionally from 2 to 7 amino acid residues, optionally from 2 to 6 amino acid residues, optionally from 2 to 5 amino acid residues, and optionally from 2 to 4 amino acid residues. In exemplary embodiments, at least a portion, or each, of said plurality of cyclic peptides comprises 2 or 3 amino acid residues. In some of any of the aforementioned embodiments, each amino acid reside is an a-amino acid residue

According to some of any of the embodiments described herein, at least one, preferably at least two, and optionally all, of the amino acid residues forming the cyclic peptide is/are aromatic amino acid residue(s), as described herein. When two or more aromatic amino acid residues are present, the aromatic amino acid residues can be the same or different.

According to some of any of the embodiments described herein, a cyclic peptide is a cyclic dipeptide, comprised of two amino acid residues. In some of these embodiments, each of the two amino acid residues is independently an aromatic amino acid residue. The two aromatic amino acid residues can be the same or different.

According to some of any of the embodiments described herein, the presence of aromatic amino acid residues in the cyclic peptide allows the plurality of cyclic peptides to self-assemble so as to form a supramolecular structure.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to, N-terminus modification, C-terminus modification, peptide bond modification, including, but not limited to, CH2-NH, CH2-S, CH2-S=0, 0=C—NH, CH2-0, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992). Peptide bonds (—CO—NH—) within the peptide may be substituted, for example, by N-methylated bonds (—N(CH3)-CO—), ester bonds (—C(R)H—C-0-0-C(R)—N—), ketomethylene bonds (—CO—CH2-), a-aza bonds (—NH—N(R)—CO—), wherein R is any alkyl, e.g., methyl, carba bonds (—CH2-NH—), hydroxyethylene bonds (—CH(OH)—CH 2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom. These modifications can occur at any of the bonds along the peptide chain and even at several (2-3) at the same time.

In any of the respective embodiments herein pertaining to a cyclic peptide, each of the amino acid residues of the cyclic peptide may independently be a coded amino acid residue or a non-coded amino acid residue. Herein, a “coded” amino acid refers to any of the 20 “standard” amino acids encoded by the universal genetic code.

As used herein throughout, the term “amino acid” or “amino acids” is understood to include the 20 naturally occurring amino acids, which are also referred to herein as “coded” amino acids; those amino acids often modified post-translationally in vivo, including, for example, hydroxyproline, phosphoserine and phosphothreonine; and other unusual amino acids, including synthetically prepared amino acids, including, but not limited to, 2-aminoadipic acid, hydroxylysine, isodesmosine, nor-valine, nor-leucine and ornithine. The term “amino acid” includes both D- and L-amino acids.

Natural aromatic amino acids, Trp, Tyr, His and Phe, may be substituted for synthetic unnatural acids such as phenylglycine, TIC, naphthylalanine (Nal), ring-methylated derivatives of Phe, halogenated derivatives of Phe or O-methyl-Tyr, imidazole-substituted derivatives of His, and b amino-acids. Such modified amino acids are also referred to herein as structural analogs of the aromatic amino acids.

The cyclic peptides described herein can include any combination of: cyclic dipeptides composed of one or two aromatic amino acid residues; cyclic tripeptides including one, two or three aromatic amino acid residues; cyclic tetrapeptides including two, three or four aromatic amino acid residues; cyclic pentapeptides including two, three, four or five aromatic amino acid residues; and cyclic hexapeptides including two, three, four, five or six aromatic amino acid residues. The phrase “aromatic amino acid residue”, as used herein, refers to an amino acid residue that comprises an aromatic moiety in its side-chain.

As used herein, the phrase “aromatic moiety” describes a monocyclic or polycyclic moiety having a completely conjugated pi-electron system. The aromatic moiety can be an all-carbon moiety (aryl) or can include one or more heteroatoms such as, for example, nitrogen, sulfur or oxygen (heteroaryl). The aromatic moiety can be substituted or unsubstituted, whereby when substituted, the substituent can be, for example, one or more of alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano and amine. The aromatic moiety can include one or more aryl and/or heteroaryl groups, as defined hereinbelow, which can be fused or non-fused to one another.

Exemplary aromatic moieties include, but are not limited to, phenyl, biphenyl, naphthalenyl, phenanthrenyl, anthracenyl, [1,10]phenanthrolinyl, indoles, imidazoles, thiophenes, thiazoles and [2,2′]bipyridinyl, each being optionally substituted. Thus, representative examples of aromatic moieties that can serve as the side chain within the aromatic amino acid residues described herein include, without limitation, substituted or unsubstituted naphthalenyl, substituted or unsubstituted phenanthrenyl, substituted or unsubstituted anthracenyl, substituted or unsubstituted [1,10]phenanthrolinyl, substituted or unsubstituted [2,2′]bipyridinyl, substituted or unsubstituted biphenyl, and substituted or unsubstituted phenyl. The aromatic moiety can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine.

When substituted, the aromatic moiety includes one or more substituents such as, but not limited to, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine. Exemplary substituted phenyls may be, for example, pentafluoro phenyl, iodophenyl, biphenyl and nitrophenyl.

Herein, a “cyclic peptide” is also referred to as “cyclo-peptide”, whereby specific peptides are preceded by the prefix “cyclo-” or “cyclic”.

A cyclic peptide according to any of the respective embodiments described herein may optionally be a cyclic peptide obtainable by linking a peptide C-terminus to a peptide N-terminus by an amide bond, by linking two side-chains (e.g., cysteine side chains) by a disulfide (—S—S—) bond, by a lactam bridge, by a hydrocarbon-staple (optionally a chiral hydrocarbon staple), by a triazole bridge, by bio-Cys alkylation, or by an acetone Hey linker, and/or by any form of peptide cyclization described in the art, e.g., in Hu et al. [Angew. Chem. Int. Ed. 55:8013-8017 (2016)]. In exemplary embodiments, a cyclic peptide as described herein is a peptide in which a peptide's C-terminus is linked to its N-terminus by an amide bond.

In some of any of the respective embodiments, at least one of the amino acid residues (in at least a portion, or each, of the plurality of cyclic peptides) comprises an aromatic moiety. In some embodiments, at least two adjacent amino acid residues each comprise an aromatic moiety. Examples of amino acid residues comprising an aromatic moiety include, without limitation, residues of phenylalanine (Phe), tyrosine (Tyr), tryptophan (Trp), histidine (His), β,β-diphenylalanine (Dip), naphthylalanine (Nal), and dihydroxyphenylalanine (DOPA).

In any of the respective embodiments herein, a cyclic peptide is a cyclic dipeptide, e.g., a substituted diketopiperazine.

Each of these cyclic dipeptides can include one or two aromatic amino acid residues. Preferably, each of these dipeptides includes two aromatic amino acid residues. The aromatic residues composing the cyclic dipeptide can be the same, such that the cyclic dipeptide is a cyclic homodipeptide, or different.

The phrase “aromatic cyclic dipeptide” as used herein describes a cyclic peptide composed of two amino acid residues, at least one, and preferably both, being an aromatic amino acid as defined herein.

According to some of any of the embodiments described herein, the aromatic cyclic dipeptide comprises in its side chain an aromatic group which is unsubstituted or which is substituted by one or more substituents as described herein.

According to some of any of the embodiments described herein, a cyclic peptide comprises aromatic homodipeptides, having two aromatic amino acid residues which are identical with respect to their side-chains residue, or in which the two aromatic amino acid residues are identical (the same). Exemplary aromatic cyclic homodipeptides include, but are not limited to, phenylalaninephenylalanine cyclic dipeptide, naphthylalanine-naphthylalanine cyclic dipeptide, (pentaflurophenylalanine)-(pentafluro-phenylalanine) cyclic dipeptide, (iodo-phenylalanine)-(iodophenylalanine) cyclic dipeptide, (4-phenyl phenylalanine)-(4-phenyl phenylalanine) cyclic dipeptide, (p-nitro-phenylalanine)-(p-nitro-phenylalanine) dipeptide, tryptophan-tryptophan cyclic dipeptide, tyrosine-tyrosine cyclic dipeptide, and histidine-histidine cyclic dipeptide.

According to some of any of the embodiments described herein, each of the aromatic homodipeptides is an unsubstituted tryptophan-tryptophan dipeptide (cyclo-Trp-Trp; cyclo-WW). In some of these embodiments, the self-assembled structure is in a form of a nanospheres (e.g., a plurality of nanospheres).

According to some of any of the embodiments described herein, at least a portion of, or each cyclic peptide in, the plurality of aromatic cyclic dipeptides comprises aromatic heterodipeptides, having two aromatic amino acid residues which are different with respect to their side-chains residue, or in which the two aromatic amino acid residues are different with respect to their chirality. Exemplary such aromatic cyclic dipeptides include, but are not limited to, phenylalaninetryptophan cyclic dipeptide, naphthylalanine-tryptophan cyclic dipeptide, (pentaflurophenylalanine)-tryptophan cyclic dipeptide, (iodo-phenylalanine)-tryptophan cyclic dipeptide, (4-phenyl phenylalanine)-tryptophan cyclic dipeptide, (p-nitro-phenylalanine)-tryptophan dipeptide, phenylalanine-tyrosine cyclic dipeptide, naphthylalanine-tyrosine cyclic dipeptide, (pentaflurophenylalanine)-tyrosine cyclic dipeptide, (iodo-phenylalanine)-tyrosine cyclic dipeptide, (4-phenyl phenylalanine)-tyrosine cyclic dipeptide, (p-nitro-phenylalanine)-tyrosine dipeptide, phenylalanine-histidine cyclic dipeptide, naphthylalanine-histidine cyclic dipeptide, (pentaflurophenylalanine)-histidine cyclic dipeptide, (iodo-phenylalanine)-histidine cyclic dipeptide, (4-phenyl phenylalanine)-histidine cyclic dipeptide, (p-nitro-phenylalanine)-histidine dipeptide, tryptophan-histidine cyclic dipeptide, tyrosine-tryptophan cyclic dipeptide, and histidine-tyrosine cyclic dipeptide.

According to some of any of the embodiments described herein, each of the aromatic cyclic dipeptides comprises a (substituted or unsubstituted) imidazole in its side chain.

In some of any of the embodiments described herein, for any of the above-mentioned aromatic cyclic dipeptides, each of the amino acid residues is L-amino acid residue. In some of any of the embodiments described herein, for any of the above-mentioned aromatic cyclic dipeptides, each of the amino acid residues is D-amino acid residue. In some of any of the embodiments described herein, for any of the above-mentioned aromatic cyclic dipeptides, one of the amino acid residues is D-amino acid residue and one of the amino acid residues is L-amino acid residue.

In some embodiments, all of the aromatic cyclic dipeptides in the plurality of cyclic peptides forming the self-assembled structures are the same, that is, all have the same amino acid residues, and the same type of peptide bond linking therebetween. In some of these embodiments, the amino acids residues have the same or different chirality.

According to an aspect of some embodiments of the present invention the self-assembled structure is formed of a plurality of cyclic peptides as described herein that comprises or consists of a plurality of cyclo-WW, or a plurality of cyclo-WF, or a plurality of cyclo-YY or a plurality of cyclo-FF. According to an aspect of some embodiments of the present invention the self-assembled structure is formed of a plurality of cyclic peptides as described herein that comprises or consists of a plurality of cyclo-WW.

According to an aspect of some embodiments of the present invention the self-assembled structure is formed of a plurality of cyclic peptides as described herein that comprises a plurality of cyclic aromatic dipeptides as described herein, and at least a portion, or each, of the plurality of cyclic dipeptides comprises or consists of cyclic aromatic dipeptides (heterodipeptides or homodipeptides) as described herein, each comprising one aromatic amino acid that comprises an imidazole in its side-chain (e.g., histidine).

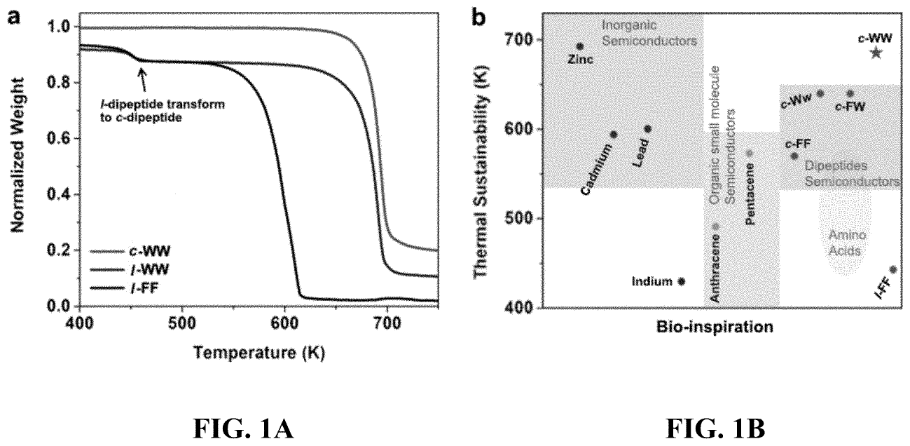

The present inventors found that simple aromatic dipeptides, and in particular, an exemplary cyclo-ditryptophan (cyclo-WW or c-WW), self-assembles into supramolecular semiconductors with high thermal sustainability. FIGS. 1A-1B present a thermal sustainability characterization of an exemplary cyclo-dipeptide according to some embodiments of the present invention, cyclo-WW. FIG. 1A shows thermal gravimetric analysis (TGA) curves of cyclo-WW and linear-WW. The TGA curve of FF crystals was added for comparison. It is noted that at ˜440 K (marked by arrow), linear dipeptides transformed into cyclic ones following the removal of a water molecule. FIG. 1B shows a comparison of the thermal sustainability of state-of-the-art semiconductive constituents. The thermal sustainability of inorganic elements, zinc, cadmium, lead and indium, was added for comparison. Data for Zinc, Cadmium, Lead and Indium was used from S. W. Holman, R. R. Lawrence, L. Barr, in Proceedings of the American Academy of Arts and Sciences, Vol. 31, JSTOR, 1895, pp. 218-233. Data for Anthracene and Pentacene was taken from C. Enengl, S. Enengl, M. Havlicek, P. Stadler, E. D. Glowacki, M. C. Scharber, M. White, K. Hingerl, E. Ehrenfreund, H. Neugebauer, N. S. Sariciftci, Adv. Funct. Mater. 2015, 25, 6679-6688; and M. Sytnyk, E. D. Glowacki, S. Yakunin, G. Voss, W. Schöfberger, D. Kriegner, J. Stangl, R. Trotta, C. Gollner, S. Tollabimazraehno, G. Romanazzi, Z. Bozkurt, M. Havlicek, N. S. Sariciftci, W. Heiss, J. Am. Chem. Soc. 2014, 136, 16522-16532. Data for cyclo-FW was taken from K. Tao, B. Xue, Q. Li, W. Hu, L. J. Shimon, P. Makam, M. Si, X. H. Yan, M. J. Zhang, Y. Cao, R. Yang, J. B. Li, E. Gazit, Mater. Today 2019, 30, 10-16. Data for amino acids was taken from J. Clark, https://www.chemguide.co.uk/organicprops/aminoacids/background.html#:˜:text=The %20amino%20acids%20are%20crystalline,200%20%2D%20300%C2%B0C%20range., 2004.

Thermal gravimetric analysis (TGA) demonstrated that the c-WW crystals could sustain up to 680 K in an argon gas environment (FIG. 1A, red curve), 110 K higher than the extensively studied diphenylalanine (FF) system (570 K, FIG. 1A, black curve) and 100 K-200 K higher than the amino acids ones (FIG. 1B). Thus, the c-WW crystals showed superior thermal sustainability compared to inorganic substances, such as cadmium (574 K) or lead (601 K), and similar to zinc (693 K) (FIG. 1B). To validate this finding, linear-WW (l-WW) crystals were also investigated, showing the same weight loss curve as c-WW after the phase transition point at 440 K, where the liner dipeptides transformed into the cyclic counterparts (FIG. 1A, blue curve). These findings unexpectedly demonstrate the c-WW self-assemblies to be the most thermo-sustainable peptide system reported so far, significantly raising the heating upper-limit of organic semiconductors.

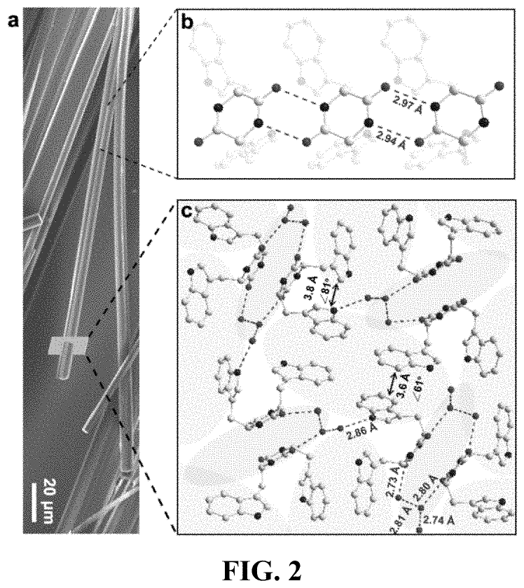

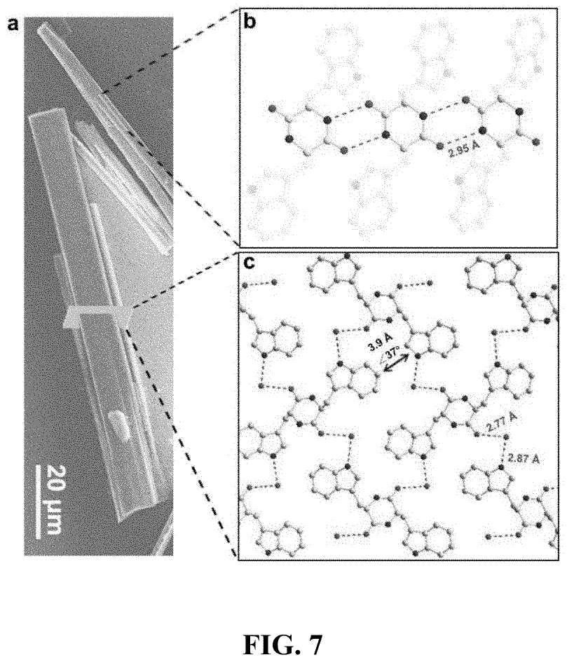

The present inventors found that cyclo-dipeptides are more inclined to organize into superstructures compared to the linear counterparts due to the lack of amino or carboxylic groups. Aromatic cyclo-dipeptides can self-assemble into supramolecular architectures through aggregation-induced quantum confinement in the hydrogen bonding (H-bonds) and aromatic ring regions. The present inventors have previously found that c-WW could oligomerize into nanodots, which showed quantum-confined photoluminescence in the visible light region. Following packing, the dots further organized into needle-like crystals (FIGS. 2 and 7).

FIG. 2 illustrates crystallographic characterization of the cyclo-WW crystals, which shows two types of extensive H-bonds networks in the assemblies. Section (a) of FIG. 2 shows a SEM image of needle-like crystals. Section (b) of FIG. 2 shows crystal structure along the crystal axis direction. For clarity, the side-chain indole rings are blurred. Section (c) of FIG. 2 shows a crystal structure along the crystal transection. The H-bonds and π-π regions are designated in light red and light blue, respectively. The hydrogen bonding and aromatic interactions are labelled near their respective locations. Data is Sections (b) and (c) is adapted with permission from K. Tao, B. Xue, Q. Li, W. Hu, L. J. Shimon, P. Makam, M. Si, X. H. Yan, M. J. Zhang, Y. Cao, R. Yang, J. B. Li, E. Gazit, Mater. Today 2019, 30, 10-16.

The first formed parallel β-sheets along the diketopiperazine rings (the crystal axis direction), with N_{backbone} . . . O_{backbone} (donor . . . acceptor) distances of 2.97 Å and 2.94 Å (FIG. 2, Section (b)), while the other was positioned along the transversal direction of the crystal, combining the adjacent backbones through two pairs of water molecules to form a circular chain, comprising three H-bonds of two O_{water} . . . O_{backbone} and one O_{water} . . . O_{water} distances of 2.80 Å, 2.73 Å, and 2.81 Å, respectively (FIG. 2, Section (c), light red region). Simultaneously, the side-chain aromatic indole rings organized into intermolecular “edge-to-face” π-π interactions, with a shortest atomic distance of 3.6 Å and a dihedral angle of 61° (FIG. 2, Section (c), light blue region). Therefore, the present inventors discovered that the c-WW crystals are composed of alternating H-bonds and π-π domains, interconnected through a water molecule on each side of the circle which is linked with the indole ring by forming two H-bonds of O_{water} . . . O_{water} (2.74 Å) and N_{indole} . . . O_{water} (2.86 Å), and an intramolecular edge-to-face π-π interaction with a nearest atomic distance of 3.8 Å and a dihedral angle of 81° (FIG. 2, Section (c)).

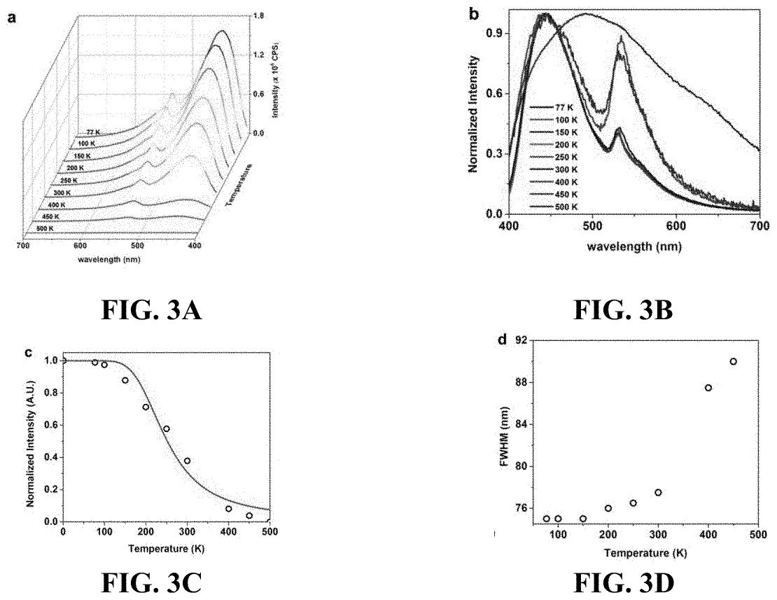

FIG. 3A illustrates a temperature-dependent emission spectrum of the of the cyclo-WW crystals excited at 370 nm. FIG. 3B shows a normalized emission spectrum shown in FIG. 3A. FIG. 3C shows a temperature-dependent normalized integrated intensity of the zero-phonon emission line at 445 nm (77 Kelvin). The red solid line represents the best-fit curve obtained using Equation 2 below. FIG. 3D shows FWHM of zero-phonon emission line evolution versus temperature.

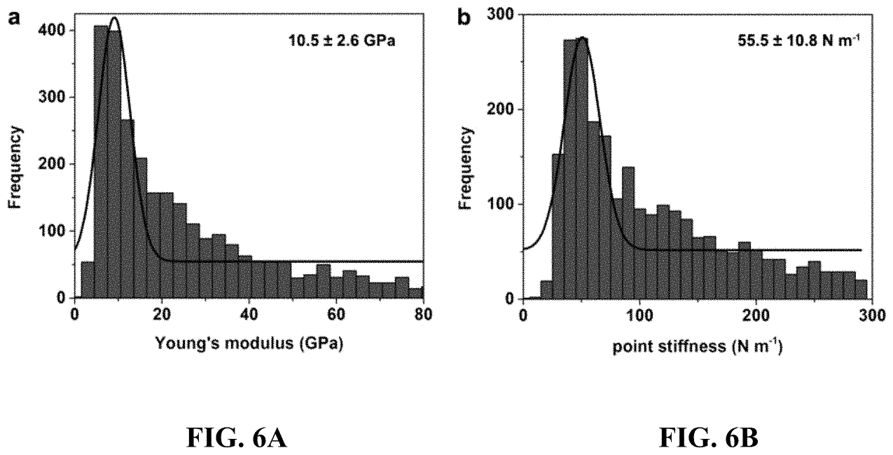

The extensive and compact integration of the non-covalent driving forces resulted in a wide-spectrum photoluminescence of the c-WW crystals, showing a maximal emission at 440 nm along with a satellite peak at 530 nm (FIGS. 3A-3B), as well as in stiff mechanical rigidity, with a measured Young's modulus of 10.5±2.6 GPa and point stiffness of 55.5±10.8 N m⁻¹ (FIGS. 6A-6B). It was discovered, unexpectedly, that the photoluminescence was remarkably affected by the temperature. As shown in FIG. 3A, the emission in the air gradually attenuated with the temperature increasing above 77 K, until transforming into a new emission with a maximal peak of 490 nm at 500 K due to the oxidation of the side-chain indole rings by atmospheric oxygen (FIG. 3B). It was discovered that the high temperature likely increases the motion freedom degree of the molecules, resulting in exacerbated non-radiative recombination, thus inducing thermal quenching of the photoluminescence.

The exciton binding energy (Eₓ) was determined by the difference between the bandgap energy (E_{g}=3.09 eV) and the position of the broad emission peak (443 nm, E_{b}=2.80 eV) using equation (1) below:

Eₓ=E_{g}−E_{b} (1)

The obtained exciton binding energy Eₓ=0.29 eV was significantly smaller than that of FF (0.34 eV). This lower binding energy induces the less localized behavior of the excitons, thus accounting for the long-wavelength (low energy) emission in the visible light region.

To obtain further insight into the excitonic process in the c-WW crystals, the present inventors plotted the zero-phonon line (ZPL) intensity (the high-energy peak in FIG. 3B) versus temperature, as shown in FIG. 3C. Equation (2) below can be used to describe thermal quenching of the excitonic luminescence:

$$I ( T ) = \frac{{I}_{0}}{1 + {Ae}^{( - {E}_{a} / {k}_{B} T )}} \qquad ( 2 )$$

where Tis the absolute temperature, I₀ is the intensity at T=0 K, A=τ_{B}/τ₀ (τ is the radiative lifetime), E_{α} is the activation energy of thermal quenching, and k_{B} is the Boltzmann constant.

By fitting the experimental results, the thermal quenching energy Eₐ=0.11±0.017 eV was obtained, 5-fold higher than that of FF (0.021±0.002 eV). Furthermore, the thermal dependence of the full-width-at-half-maximum (FWHM) of the ZPL showed, unexpectedly, that for the c-WW crystals, the start-point of the FWHM increase was approximately 300 K (FIG. 3D), significantly higher than that of FF (100 K-150 K). This shows that more heat is needed to generate thermal quenching in the c-WW crystals, thus demonstrating their high thermal sustainability.

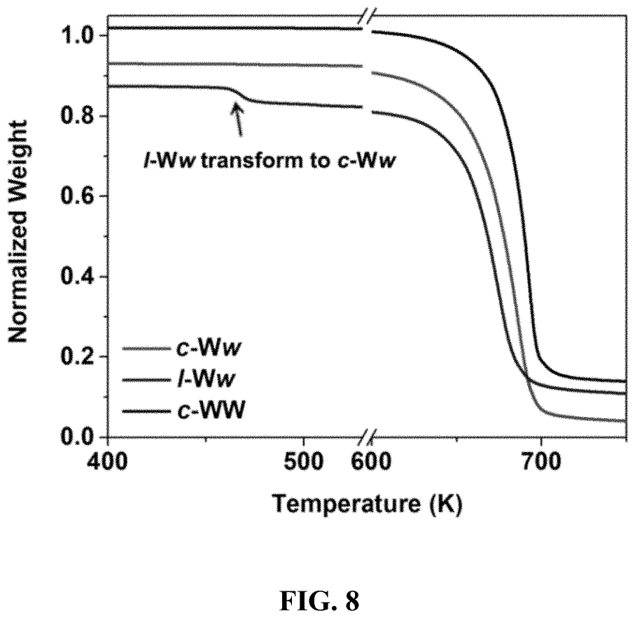

To investigate the effect of the extensive non-covalent interactions on the thermal sustainability, cyclo-tryptophan-(D)-tryptophan (c-Ww) was designed. Crystallographic characterization demonstrated that the c-Ww crystals were organized through weaker “parallel displayed” π-π interactions and inferior H-bonds networks compared to the c-WW system (FIG. 7). Correspondingly, TGA measurements demonstrated that the c-Ww packings were destructed at approximately 630 K (FIG. 8), 50 K lower than c-WW crystals, thus confirming that the extensive distribution of the non-covalent interactions underlies the high thermal sustainability.

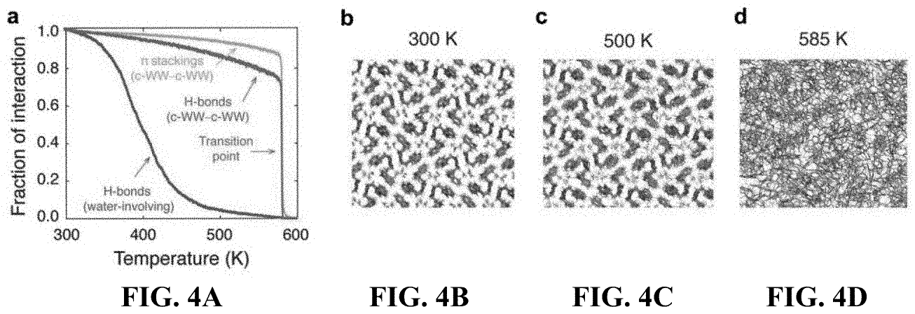

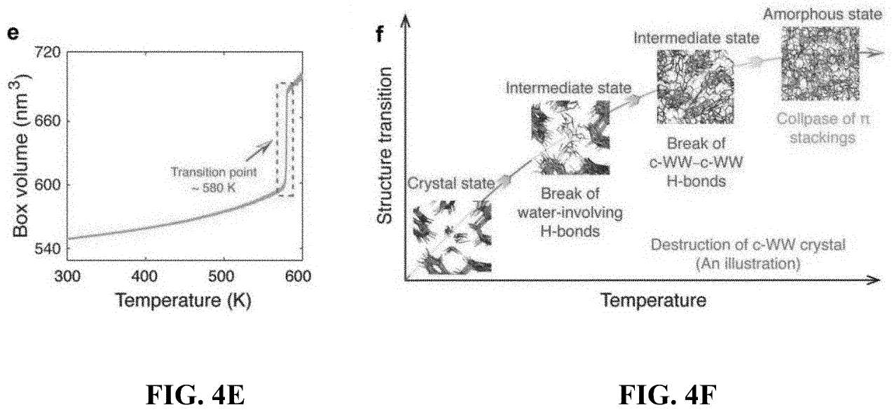

FIGS. 4A-4F illustrate a microscopic mechanism of the high thermo-sustainability of the cyclo-WW crystals. FIG. 4A shows the interaction fractions inside the cyclo-WW crystals versus the temperature. FIGS. 4B-4D illustrate snapshots of part of the system at 300 Kelvin, 500 Kelvin, and 585 Kelvin. FIG. 4E shows the volume of the simulation box plotted versus the temperature. FIG. 4F is a schematic illustration showing the dynamic process of the destruction of cyclo-WW crystal structures upon temperature increase.

Molecular dynamics simulations were further performed on the c-WW crystal structure containing 250 unit-cells. The system was gradually heated from 300 K to 600 K with a heating speed of 5 K ns⁻¹. As shown in FIG. 4A, the fraction of water-mediated H-bonds decreased quickly and showed a phase transition-like behavior at approximately 400 K upon increasing the temperature. By contrast, more than 90% of the non-covalent interactions among c-WW molecules were retained. Until 500 K, most water-mediated H-bonds were eliminated, whereas 85% of the H-bonds and 95% of the π-π stacking interactions among c-WW molecules were still intact (FIG. 4A). This resulted in the crystallized water molecules deviating away from their original lattice positions, while the supramolecular architectures persisted (FIGS. 4B-4C). When the temperature further increased up to 580 K, an abrupt drop was observed in the fractions of c-WW H-bonds and π-π stacking, from 75% and 90%, respectively, to nearly 0% (FIG. 4A), demonstrating the transition of the crystal structures to amorphous states (FIG. 4D).

Notably, the calculated transition temperature was lower than that measured using TGA, due to the fact that the parameters of water molecules used in the simulations were derived from the bulky water system, while the crystallized water molecules are actually in interfacial states, which can form stronger H-bonds compared to the bulky ones. This structural transition was accompanied by an increase of the simulation box volume, from 544 nm³ at 300 K to 570 nm³ at 500 K and 690 nm³ at 580 K (FIG. 4E). Notably, during the entire simulation period, the fraction of the π-π stacking interactions was constantly the highest, followed by H-bonds among c-WW molecules and then the water-mediated ones. The results demonstrate that π-π stacking is of paramount importance underlying the high thermal sustainability of the c-WW crystals.

Therefore, the present inventors have discovered that upon temperature rise, the water-mediated H-bonds are first distorted, followed by an increase of motion freedom degree of the H-bonds among c-WW molecules. As temperature continues to rise, the aromatic π-π interactions collapse, which finally cannot counterbalance the absorbed heat energy and the crystals are eventually destructed (FIG. 4F).

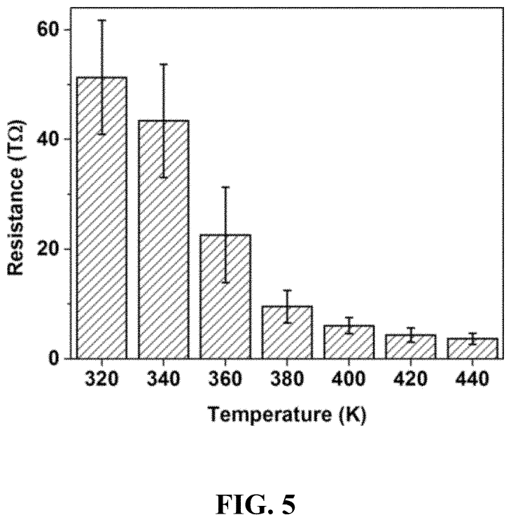

The considerable thermal sustainability endows the c-WW crystals the potential to be used for heat-stimulated applications. To further substantiate this option, temperature-reliant conductivity characterization was performed, demonstrating that the resistance of the c-WW crystals significantly declined as the temperature increased, with 93% reduction from 51.3±10.4 TΩ at 320 K to 3.6±0.9 TΩ at 440 K, as shown in FIG. 5, a characteristic of bio-organic, wide-gap semiconductors.

The present inventors thus discovered, unexpectedly, that cyclo dipeptide assemblies, and in particular, tryptophan-based cyclo-dipeptide self-assemblies, show high thermal sustainability. Non-covalent aromatic interactions and H-bonds among the peptide molecules, as well as water-mediated H-bonds, in this order, underlie this property. The thermal sustainability induces the bioinspired supramolecular architectures to show a temperature-dependent conductivity. The present inventors further discovered that peptide self-assemblies with much higher thermal resistance can be developed through rational design of the packings by tuning the non-covalent interactions.

These discoveries present thermo-sustainable peptide self-assemblies, paving the way for developing bioinspired supramolecular organizations for applications in heat-sensitive fields, such as body temperature detection and heat harvesting in bio-integrated microdevices. The peptide-based thermo-sustainable materials are a useful platform for heat-sensitive solid-state optical and electrical applications. The environmental-friendly nature of this system makes it attractive for application in body-temperature detection or wasting heat recycling in smart devices such as smartphones and self-charging power packages for sustainably operating mobile or wearable electronics. The inventive peptide-based thermo-sustainable materials are particularly suited for use in wearable smart devices, such as smartwatches and smartphones, for monitoring body temperature fluctuation in daily lives or running, riding and using gymnastic equipment. Because of the environmentally friendly nature of the inventive peptide materials, the nanostructures can be used as a permanently self-powered device for medical implant systems.

Experimental Data:

Materials. Dipeptides were purchased from Bachem (Bubendorf, Switzerland), GL Biochem (Shanghai, China) or DgPeptides (Hangzhou, China). Water was processed by a Millipore purification system (Darmstadt, Germany) with a minimum resistivity of 18.2 MΩcm.

Crystals preparation. The peptide powders were dissolved in water to a concentration of 1.0 mM. The solutions were then incubated in an 80° C. water bath for 10 min, followed by filtration using 0.45 PVDF membranes (Merck Millipore, Carringtwohill, Ireland) and pH adjustment to 7.0±0.2. Subsequently, needle-like crystals appeared and reached maximum size after 30 days. The solutions were centrifuged, the crystals were washed three times with water and then collected for later use.

Scanning electron microscopy (SEM). The solution containing the crystals was placed onto a clean glass slide, allowed to adsorb for a few seconds and excess liquid was removed using a filter paper. The slide was then coated with Cr and observed under a JSM-6700 field emission scanning electron microscope (JEOL, Tokyo, Japan) operated at 10 kV.

Temperature-dependent fluorescent emission. For fluorescent emission characterization, the crystals were stressed to form a film on a clean aluminum substrate, and the spectra were collected on a FluoroMax-4 Spectrofluorometer (Horiba Jobin Yvon, Kyoto, Japan). The excitation wavelength was set at 370 nm with a slit of 5 nm, the emission wavelength was set at 350-800 nm with a slit of 5 nm and a step of 2 nm. The substrate temperature was gradually increased from 77 K to 500 K.

Thermal gravimetric analysis (TGA). TGA experiments were performed using a TA Instruments (USA) module SDT 2950, at a temperature range between 300 K and 800 K with a heating rate of 10 K min-1, under dry ultrahigh-purity argon atmosphere.

Computational simulation of temperature-evolved crystal box volume. All simulations were performed in the isothermal-isobaric ensemble using the Gromacs 2016.4 package. See M. J. Abraham, T. Murtola, R. Schulz, S. Pall, J. C. Smith, B. Hess, E. Lindahl, SoftwareX2015, 1, 19-25. The simulation box was built by repeating the crystal unit-cell 10 times, 5 times, and 5 times in the a, b, and c direction, respectively. The force field for c-WW molecules was built based on atom types in OPLS force field with electrostatic interaction parameters fitted by quantum mechanical calculations, while force field for water molecules was taken from TIP-4P model. See W. L. Jorgensen, J. Tirado-Rives, J. Am. Chem. Soc. 1988, 110, 1657-1666; J. L. F. Abascal, C. Vega, J. Chem. Phys. 2005, 123, 234505. The system contained 250 unit-cells (consisting of 1000 c-WW and 3000 water molecules). The pressure was kept at 1 bar using the Berendsen method with a coupling time constant of 1.0 ps. See H. J. C. Berendsen, J. P. M. Postma, W. F. v. Gunsteren, A. DiNola, J. R. Haak, J. Chem. Phys. 1984, 81, 3684-3690. The temperature of the system was controlled by coupling all atoms to a single external heat bath using a V-Rescaling coupling method (with a relaxation time of 0.1 ps). See G. Bussi, D. Donadio, M. Parrinello, J. Chem. Phys. 2007, 126, 014101. The reference temperature was 300 K at t=0, and was linearly increased to 600 K at t=60 ns. Constraints were applied to all bond lengths using the LINCS method. See B. Hess, J. Chem. Theory Comput. 2008, 4, 116-122. Electrostatic interactions were treated with the Particle Mesh Ewald method with a real space cutoff of 1.2 nm. See T. Darden, D. York, L. Pedersen, J. Chem. Phys. 1993, 98, 10089. Van der Waals interactions were calculated using a cutoff of 1.2 nm. Simulations were conducted using periodic boundary conditions.

Hydrogen bonds were defined using a cutoff of 150° for the angle (Donor-Hydrogen-Acceptor) and 0.35 nm for the distance (Donor-Acceptor). Two phenyl rings were considered to form π-π stacking interactions if their centroid distance was shorter than 0.7 nm. See S. K. Burley, G. A. Petsko, Science 1985, 229, 23-28. All analyses were performed using tools implemented in Gromacs and in-house developed codes.

Young's modulus measurement. Atomic force microscopy (AFM) experiments were carried out using a commercial AFM (JPK, Nanowizard II, Berlin, Germany). The force curves were obtained using the commercial software from JPK and analyzed by a custom-written procedure based on Igor pro 6.12 (Wavemetrics Inc.). Silica cantilevers (SSS-SeIHR-50 Nanosensor Company with a half-open angle of pyramidal face of Θ<10°, tip radius: 2-10 nm, frequency in air: 96˜175 kHz) were used in all experiments. The spring constant of the cantilevers was in the range of 10˜130 N m⁻¹. The maximum loading force was set at 150 nN. All AFM experiments were carried out at room temperature. In a typical experiment, the c-WW crystals were cast on the surface of the glass substrate and the cantilever was moved over the crystal at a constant speed of 15 μm s⁻¹ guided by an optical microscope. The cantilever was held on the crystal surface at a constant force of 150 nN. Then, the cantilever was retracted and moved to another spot for the next cycle. The indentation fit was performed using an Igor custom-written program and manually checked after the fitting was completed. The curves were then fitted manually. Each approaching force-deformation curve was fitted in the range of 10 nm from the contact point, or from the maximum indentation depth to the contact point if the former was less than 10 nm. By fitting the approaching curve to the Hertz model (3) below, the present inventors obtained the Young's modulus of the c-WW crystals. Typically, 5-8 such regions (10×10 μm, 600 pixels) were randomly selected on each crystal to construct the elasticity histogram.

$$F ( h ) = \frac{2}{π} tan α \frac{{E}_{peptide}}{1 - {v}_{peptide}^{2}} {h}^{2} \qquad ( 3 )$$

where F is the stress of the cantilever, h is the depth of the c-WW crystal pressed by the cantilever tip, a is the half angle of the tip, E is the Young's modulus of the crystal and ν is the Poisson ratio. The present inventors chose ν=0.3 in the calculations.

Point stiffness calculation. The measured point stiffness (kₘₑₐₛ) is comprised of the stiffness constants of the cantilever (k_{can}) and the crystals (k_{cry}). Assuming that the c-WW crystal and the cantilever act as two springs oriented in a series, the point stiffness of the c-WW crystal could be calculated using the following relation: Using equation (4) below and an averaged measured value for kₘₑₐₛ, the average stiffness of the c-WW crystal could be calculated. To estimate the material property of the crystals, it was assumed that the mechanical behavior of the c-WW crystal could be described as linear elastic, which is a good approximation for solids under small strains.

$${k}_{cry} = \frac{{k}_{can} \cdot {k}_{meas}}{{k}_{can} - {k}_{meas}} \qquad ( 4 )$$

FIGS. 6A-6B illustrate mechanical characterization of exemplary cyclo-dipeptide featuring a diketopiperazine skeleton according to some embodiments of the present invention, cyclo-WW. FIG. 6A shows Young's modulus and FIG. 6B shows Point stiffness of cyclo-WW crystals. The normal distribution curves are also shown (black). At least 2500 counts were used for statistics

Conductivity measurement at different temperatures. The c-WW crystals were spread on a SiO₂ substrate on a cooling-heating stage with coated Au parallel electrodes. Tungsten needle electrodes were gently moved to contact the parallel electrodes under an optical microscope. The voltage (5 V) was applied using a digital power and the I-V curves were recorded while the temperature of the substrate was increased at a rate of 2 K min-1. At least five samples were tested at each temperature and averaged for accuracy.

FIG. 7 illustrates crystallographic characterization of c-Ww crystals. Section (a) shows a SEM image of needle-like crystals assembled by c-Ww. Section (b) shows a crystal structure along the axis direction of the. For clarity, the side-chain indole rings are blurred. Section (c) shows a crystal structure along the transection of the crystal. The hydrogen bonding and aromatic interactions are labelled near their respective locations.

The indole rings organized into intermolecular “parallel displayed” π-π interactions with a shortest atomic distance of 3.9 Å and a dihedral angle of 37° in the transversal direction of the c-Ww crystals, significantly weaker than the “edge-to-face” counterparts in the c-WW system. Adjacent c-Ww monomers were interconnected by a single water molecule via two Owater . . . Obackbone and Nindole . . . Owater H-bonds with distances of 2.77 Å and 2.87 Å, respectively. Therefore, the non-covalent interactions in the c-Ww crystals are inferior to those in the c-WW ones.

FIG. 8 shows TGA curves of c-Ww (shown in red) and l-Ww (shown in blue). The TGA curve of cyclo-WW (shown in black) was extracted from FIG. 3 discussed above for comparison. Note that at ˜440 K (marked by arrow), l-Ww molecules transform into c-Ww due to intramolecular condensation following the removal of a water molecule.

The TGA curve of the l-Ww crystals showed the same weight loss trace observed for c-Ww crystals after the phase transition point at 440 K, thus confirming the accuracy of the thermal sustainability of the c-Ww crystals up to 630 K.

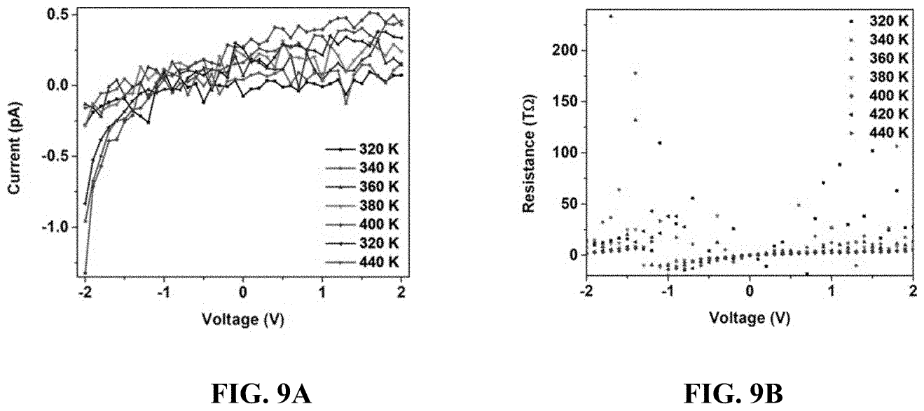

FIGS. 9A-9B illustrate temperature-dependent conductivity of the cyclo-WW crystals. FIG. 9A shows voltage-current curves of cyclo-WW crystals at different temperatures. FIG. 9B shows resistance distribution calculated from FIG. 9A.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

1. A thermally stable composition comprising at least one aromatic cyclic di-peptide, wherein the composition has a thermal sustainability of up to 680 Kelvin.

2. The composition of claim 1, wherein said at least one aromatic cyclic dipeptide is a simple aromatic cyclic dipeptide.

3. The composition of claim 2, wherein said at least one simple aromatic dipeptide is a cyclo-ditryptophan.

4. The composition of claim 1, wherein the composition is a nanostructure composition comprising monomers of the at least one aromatic cyclic di-peptide.

5. The composition of claim 1, wherein said composition has a thermal sustainability of about 580 Kelvin to about 680 Kelvin.

6. The composition of claim 1, wherein said composition has a thermal sustainability of about 630 Kelvin to about 680 Kelvin.

7. The composition of claim 1, wherein said composition has a thermal sustainability of about 650 Kelvin to about 680 Kelvin.

8. The composition of claim 1, wherein said at least one aromatic cyclic dipeptide comprises a plurality of aromatic cyclic dipeptide molecules forming a self-assembled structure.

9. The composition of claim 1, wherein said composition has a thermal quenching activity energy of up to 0.11 eV.

10. The composition of claim 1, wherein said composition has a thermal quenching activity energy of about 0.03 eV to about 0.11 eV.

11. The composition of claim 1, wherein said at least one aromatic cyclic dipeptide comprises at least one indole ring.

12. The composition of claim 11, wherein said at least one indole ring comprises one or more substituent groups that modulate said thermal sustainability of the composition.

13. A semiconductor system, comprising a self-assembled structure formed of one or more cyclic peptides, wherein said at least one of said one or more cyclic peptides is an aromatic cyclic dipeptide, wherein said self-assembled structure is thermally stable at up to 680 Kelvin.

14. The semiconductor system of claim 13, wherein said one or more cyclic peptides is a cyclo-ditryptophan.

15. The semiconductor system of claim 13, wherein said self-assembled structure has a thermal sustainability of about 580 Kelvin to about 680 Kelvin.

16. The semiconductor system of claim 13, wherein said self-assembled structure has an average size of less than 100 nm at least in one dimension or cross-section.

17. An in-vivo implantable system, comprising a self-assembled structure formed of one or more aromatic cyclic dipeptides, wherein said self-assembled structure is thermally stable at up to 680 Kelvin, and wherein said implantable system is configured to self-charge.

18. The implantable system of claim 17, wherein said one or more aromatic cyclic dipeptides is a cyclo-ditryptophan.

19. A thermally stable optical system, comprising a self-assembled structure formed of the composition of claim 1.

20. The optical system of claim 19, wherein said one or more aromatic cyclic dipeptides is a cyclo-ditryptophan.
